(19) United States
(12) Patent Application Publication
(10) Pub. No.: US 2021/0332417 A1
(43) Pub. Date: Oct. 28, 2021

(54) Molecular Marker For Identifying Sanxan Gum and Its Synthetic Strain, Preparation Method And Application Thereof

(71) Applicant: HEBEI XINHE BIOCHEMICAL CO., LTD., Xingtai (CN)

(72) Inventors: Yu ZHANG, Xingtai (CN); Guopei ZHANG, Jizhou (CN); Shaohua ZHANG, Xingtai (CN)

(21) Appl. No.: 16/984,878

(22) Filed: Aug. 4, 2020

(30) Foreign Application Priority Data
Apr. 27, 2020 (CN) 202010346628.2

(51) Int. Cl. C12Q 1/689

(52) U.S. Cl. CPC C12Q 1/689

(57) ABSTRACT

The present invention relates to microorganism identification, and more particularly to a molecular marker for identifying Sanxan gum and its synthetic strain, and preparation method and application thereof. The molecular marker is prepared by PCR amplification using genomic DNA of Sphingomonas sp. with deposit number CGMCC No. 1650 or CGMCC No. 19480 as a template for PCR amplification, and the primer 1 with nucleotide sequence shown in SEQ No. 5 and the primer 2 with nucleotide sequence shown in SEQ No. 6 as primers. The present invention overcomes the problems that three gums must be separated from each other by cloning and sequencing and other operations in a compound gum system containing Sanxan gum, gellan gum and welan gum which results in cumbersome steps.

### TECHNICAL FIELD

The present invention relates to the field of identification of microorganism, and more particularly to a molecular marker for identifying Sanxan gum and its synthetic strain, and preparation method and application thereof.

### BACKGROUND

Food gums are composed of polysaccharides and their composites, which can form viscous and gelatinous polymer hydrosol after fully hydration. They plays roles of thickening, gelatinizing, suspending, etc. in food processing, so that the obtained food can have various shapes, and a soft, hard, crisp or sticky taste. Food gums can be divided into vegetable gums, animal gums, microbial gums and chemically modified gums according to their sources. Microbial gums have excellent performance, short production cycle, and is not affect by climate and geographical environment, and they have been widely used in the food fields, such as Xanthan gum synthesized by Xanthomonas campestris and Gellan gum synthesized by Sphingomonas paucimobilis, etc.

In recent years, researchers have found that a variety of microbial gums, such as welan gum, Sanxan gum, etc. can be synthesized by strains of Sphingomonas. in addition to the gellan gum. The polysaccharide main chain of these microbial gums are composed of glucose, glucuronic acid, rhamnose, and/or mannose. However, the side chain groups are diverse in type and position, and have their own unique physical and chemical properties and applications. They are collectively called sphingan Ss. Sanxan gum is also known as sphingan Ss, which is a new type of microbial gum synthesized by Sphingomonas sanxanigenens It exhibits excellent thickening, gelatinizing, emulsifying and suspending stabilizing properties. It is a new variety of sphingan Ss polymers, and is also the first microbial-derived food gum that has been independently developed and industrialized in China. Sanxan gum has passed the technical examination of the Expert Evaluation Committee of the National Health Commission according to the “Regulations on the Application and Acceptance of New Food Additive Varieties” and the “Management Method on New Food Additive Varieties” in December 2018. It is mainly used as a thickener, stabilizer and coagulant in fruit and vegetable juice (pulp) drinks, vegetable protein drinks, meat enema and other products. When food gums are used in the bioindustry, two or more components with complementary or synergistic functions are often mixed at a certain proportion according to their properties and functions of food gum components, so that countless compound gums are obtained to meet different needs for food production. Sanxan gum, welan gum and gellan gum in microbial gums are all synthesized by strains of Sphingomonas sp. However, at present, there is no quick identification method, especially for the Sanxan gum developed independently in China. A method for identifying Sanxan gum synthetic strain and a method for identifying Sanxan gum from compound gum products are needed from the perspective of production strain protection and market regulation application. With the application and promotion of Sanxan gum, and regulation of the application market of such products, how to quickly identify whether a product has been added with Sanxan gum, especially after using of compound gum, has been become an urgent problem to be solved.

The identification of Sanxan gum with polysaccharides structure is very complicated, and it is necessary to purify the polysaccharides, and then use Sminth hydrolysis, methylation analysis, and two-dimensional nuclear magnetic resonance spectroscopy and other complex steps and analysis methods. A patent document with the application number of CN201811206214.9 has disclosed “a primer of a molecular marker and molecular marker for identifying Sanxan gum, gellan gum and welan gum, and application thereof”, which can be used for identifying Sanxan gum. However, in a compound gum system simultaneously using Sanxan gum, gellan gum and welan gum, since these three microbial gums are synthesized by different strains of Sphingomonas sp., the homology of them is high, and three PCR products which are obtained by amplification of identification primers have similar sizes, so that the three gums must be separated from each other by cloning and sequencing and other operations, resulting in cumbersome steps.

### SUMMARY

The first purpose of the present invention is to provide a molecular marker for identifying Sanxan gum and its synthetic strain.

The second purpose of the present invention is to provide a pair of primers for identifying Sanxan gum and its synthetic strain.

The third purpose of the present invention is to provide a preparation method of the molecular marker for identifying Sanxan gum and its synthetic strain.

The fourth purpose of the present invention is to provide an application of the molecular marker for identifying Sanxan gum and its synthetic strain in the rapid identification and detection of Sanxan gum and its synthetic strain.

The fifth purpose of the present invention is to provide an application of the molecular marker for identifying Sanxan gum and its synthetic strain in the identification of Sanxan gum in mediumly or lightly processed products containing the Sanxan gum.

The overall technical solutions of the present invention are as follows.

A molecular marker for identifying Sanxan gum and its synthetic strain, the molecular marker being a nucleotide sequence shown in SEQ No.13.

A pair of primers for identifying Sanxan gum and its synthetic strain, the pair of primers being amplified to obtain the nucleotide sequence shown in SEQ No.13.

The pair of primers for identifying Sanxan gum and its synthetic strain, which is composed of a primer 1 and a primer 2, wherein the primer 1 is a nucleotide sequence shown in SEQ No. 5, or a nucleotide sequence obtained by deleting, and/or adding, and/or changing at least one nucleotide of the nucleotide sequence shown in SEQ No. 5; and the primer 2 is the nucleotide sequence shown in SEQ No. 6, or a nucleotide sequence obtained by deleting, and/or adding, and/or changing at least one nucleotide of the nucleotide sequence shown in SEQ No. 6.

A preparation method of the molecular marker for identifying Sanxan gum and its synthetic strain, the molecular marker comprising the pair of primers mentioned above, DNA polymerase and/or dNTPs, the preparation method comprising the following specific steps:

A, performing PCR reaction using genomic DNA of Sphingomonas sp. with deposit number CGMCC No. 1650 or CGMCC No. 19480 as a template for PCR amplification, and the primer 1 and the primer 2 as primers;

B, PCR amplification system containing 1.0 μL of 10-30 ng/μL template DNA, 0.5 μL of 10 μM primer 1, 0.5 μL of 10 μM primer 2, 1.0 μL of 10 mM dNTP, 2.5 μL 10× buffer, 0.5 μL of 5 U/μL Platinum Taq DNA polymerase, and ultrapure water to 25 μL;

PCR reaction conditions containing pre-denaturation at 95° C. for 5 minutes; 94° C. for 45 seconds, 55-63° C. for 45 seconds, 72° C. for 1 minute, 30 cycles; 72° C. extension for 10 minutes;

a PCR amplification product being about 950 bp by electrophoresis detection;

C, performing DNA sequencing of the PCR amplification product to obtain a core conserved sequence 920 bp representing Sanxan gum and its synthetic strain by removing sequences of primers at two ends that affect the accuracy of sequencing, which is a specific molecular marker for identifying Sanxan gum and its synthetic strain.

An application of the molecular marker for identifying Sanxan gum and its synthetic strain in the rapid identification and detection of Sanxan gum and its synthetic strain.

An application of the molecular marker for identifying Sanxan gum and its synthetic strain in the identification of Sanxan gum in mediumly or lightly processed products containing the Sanxan gum.

The synthetic strain of Sanxan gum is Sphingomonas sp. with Latin name of Sphingomonas sp. and a deposit number of CGMCC No. 1650 or CGMCC No. 19480.

The Sphingomonas sp. strain (CGMCC No. 19480) of the present invention has been deposited in China General Microbiological Culture Collection Center (CGMCC) on March 16, 2020, and the depository institution is located at Institute of Microbiology, Chinese Academy of Sciences, No.3, No.1 yard, Beichen West Road, Chaoyang District, Beijing.

The present invention comprises at least the following substantial improvements and beneficial effects:

the present invention screens the specific molecular marker of Sanxan gum and Sanxan gum synthetic strain-Sphingomonas sp. (CGMCC No. 1650 or CGMCC No. 19480) from the perspective of Sanxan gum synthetic gene cluster and comparative genome, and the molecular marker can identify Sanxan gum microbial gum from mediumly and lightly processed products containing the Sanxan gum, which has the advantages of short detection time and high accuracy.

### BRIEF DESCRIPTION OF THE DRAWINGS

FIG. 1 is a comparison drawing of the polysaccharide structures of Sanxan gum, welan gum, and gellan gum, wherien Glc is glucose, Man is mannose, GlcA is glucuronic acid, Rha is rhamnose, Acetyl is acetyl group, and Glyceroyl is glyceryl group.

FIG. 2 is an electrophoretic detection drawing of PCR amplification products of microbial gum synthetic strain by the primer 1 or the primer 2 in the present invention, wherein M is a molecular weight marker, 1 is a negative control; 2 is Sphingomonas sp. (CGMCC No. 1650), 3 is a gellan gum synthetic strain-Sphingomonas sp. (CGMCC No. 14238); 4 is a welan gum synthetic strain-Sphingomonas sp. (CGMCC No. 1561); 5 is a xanthan gum synthetic strain-Xanthomonas campestris (CGMCC No. 10122).

FIG. 3 is a comparison drawing of sequence of obtained highest homologous species Bosea vaviloviae strain Vaf18 and sequence of the molecular marker of the present invention when BLASTN alignment is performed between nucleic acid sequences of the molecular marker of Sphingomonas Sp. CGMCC No. 1650 or CGMCC No. 19480 with that of all species in GenBank database.

### DETAILED DESCRIPTION OF EMBODIMENTS

The present invention will be described in further detail with reference to the embodiments and the accompanying drawings below, but it is not intended to limit the present invention. The scope of protection of the present invention is based on the contents recited in the claims, and any equivalent technical means made according to the disclosure will not deviate from the scope of protection of the present invention. The methods that do not indicate specific conditions in the embodiments of the present invention are performed according to the technical means commonly used in the art or the conditions recommended by the manufacturer.

### Embodiment 1

A molecular marker for identifying Sanxan gum and its synthetic strain, the sequence of primer 1 of the molecular marker is SEQ5: 5′-TCAGGCCGTGTGGGGAA-3′, and the sequence of the primer 2 is SEQ6: 5′-GATCCGATCCAGCTTTCGGG-3′.

The method for obtaining the above primers of the molecular marker for identifying Sanxan gum and its synthetic strain includes the following steps: inoculating the Sanxan gum synthetic strain-Sphingomonas sp. CGMCC No. 1650, the gellan gum synthetic strain-Sphingomonas sp. CGMCC No. 14238; the welan gum synthetic strain-Sphingomonas sp. CGMCC No. 1561; the xanthan gum synthetic strain-Xanthomonas campestris CGMCC No. 10122 into 250 mL flasks containing 100 mL sterile liquid culture medium, respectively; the culture medium comprising (g/L): glucose 12.0; malt extract 4.0; peptone 2.5; yeast powder 1.5; pH=7.0; performing shaking cultivation at 150 rpm, 30° C. in a shaker for 48 hours; centrifuging at 6,000 rpm for 15 minutes; collecting strain cells for use; performing strain genomic DNA extraction according to the instructions of strain genomic DNA extraction kit from Tiangen Biotech (Beijing) Co., Ltd.; and freezing the obtained DNA solution at −20° C. for use.

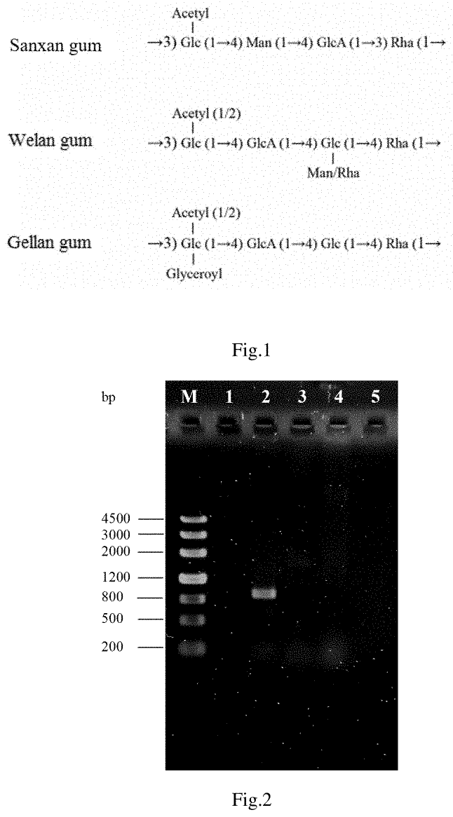

The difference between Sanxan gum and other microbial polysaccharides lies in the composition of monosaccharide, and the connection mode of the main chain and the side chain of monosaccharide. Sanxan gum, gellan gum, welan gum, and other sphingan Ss, are synthetic products of different strains of the same genus of the Sphingomonas. The composition of monosaccharides is similar, but there is mannose on the main chain of Sanxan gum, which is connected with glucose and glucuronic acid by 1,4 glycosidic bonds, respectively. The connection mode of the main chain and the side chain of monosaccharide is significantly different from the gellan gum and welan gum, as shown in FIG. 1. The corresponding glycosyltransferase and genes responsible for polysaccharide polymerization and output in the Sanxan gum synthetic gene cluster are screened according to the differences in the structure of the above polysaccharides, PCR amplification primers (as shown in the following table) are designed by combining with the comparative genomic difference analysis of Sanxan gum, gellan gum and welan gum.

| Number | Primer sequence | Number | Primer sequence |
|---|---|---|---|
| SEQ1 | TGAGGTCGAGGAACTTGTGC | SEQ7 | CAGAATGCTCGTATTGCCGC |
| SEQ2 | GTCGTGACCGACTATCGCAT | SEQ8 | TCTATCTCGACGAGCGCAAC |
| SEQ3 | AGGTCGAGGAACTTGTGCAG | SEQ9 | GGCTTCGGTGAAGAACAGGA |
| SEQ4 | TCGCCACCAAGATCTATCGC | SEQ10 | TCGATCCACCATATGCAGCC |
| SEQ5 | TCAGGCCGTGTGGGGAA | SEQ11 | CTTGGCCATATTTGCAGCCATA |
| SEQ6 | GATCCGATCCAGCTTTCGGG | SEQ12 | GACCGGATTTGCTCAGGGTT |

PCR amplification are performed by using genomic DNA of Sanxan gum synthetic strain as a template and the above-mentioned primers as primers. The PCR amplification system includes the following components: 1.0 μL of 10-30 ng/μL template DNA, 0.5 μL of 10 μM primer 1, 0.5 μL of 10 μM primer 2, 1.0 μL of 10 mM dNTP, 2.5 μL 10× buffer, 0.5 μL of 5 U/μL Taq DNA polymerase, and water to 25μL. The PCR reaction conditions includes the following conditions: pre-denaturation at 95° C. for 5 minutes; 94° C. for 45 seconds, 55-63° C. for 45 seconds, 72° C. for 1 minute, 30 cycles; 72° C. extension for 10 minutes. 5 μL of the PCR amplification product is mixed with 1 μL loading buffer. The mixture is loaded on a 1.5% agarose gel for electrophoresis, stained with GoldView nucleic acid dye for 10 min, and photographed in the gel imager. The results show that no band could be amplified. PCR amplification conditions are optimized and it is found that the primer SEQ5/6 could be amplified to a clear band around 950 bp by using Platinum Taq DNA polymerase, annealing temperature of 62° C., and genomic DNA of Sanxan gum synthetic strain as a template. Multiple bands or no band are obtained by amplification of other primers.

PCR amplification results by using SEQ5/6 as the primer and genomic DNA of Sanxan gum, gellan gum, welan gum and xanthan gum synthetic strains as templates are shown in FIG. 2. It can be seen that the PCR amplification of Sanxan gum synthetic strain can obtain a clear band about 950 bp, and no amplified band appears in the gellan gum, welan gum and xanthan gum synthetic strains, which indicates that SEQ5 and SEQ6 can be used as PCR amplification primers of specific molecular marker of the Sanxan gum synthetic strain. SEQ5 and SEQ6 are named as primer 1 and primer 2, respectively.

The PCR amplification product of the Sanxan gum synthetic strain is subjected to DNA sequencing in Beijing Aoke Dingsheng Biotechnology Co., Ltd. The sequences near primers that affect the accuracy of sequencing are removed to obtain specific molecular marker representing Sanxan gum and its synthetic strain.

The specific sequence SEQ 13 of the specific molecular marker 920 bp is as follows:

ACGGCAGGACCTCGCCTTGCAGCAGCCGCGTCGCCTGGCG
ACGGTCGAGCGCGCGCGAGAAGAGGAAGCCTTGGCCATATTTG
CAGCCATAGCGCTGCAGCAGCCGGCACTGCGCCTCCGTCTCGA
TTCCCTCGGCGACGACCCGCAGCTTGAGACCGTCGGCAATCGC
GATCAGCCCCTGCACGATCGCAGCGCTGCCCGCATCGGTGCCG
AGCTGCTGGACGAAGGAGCGGTCGATCTTGATGATGTCCACCG
GCACCGAGAGCAGGTGCGTCAGCGAGGCATAGCCGGTACCGA
AATCGTCGAGCGCGATGCGGAGCCCGCGCGCCTGCAATCCTTC
GAGAACGCGGCGCACGGTATCGGCGCGCCGGTCCATATGGACC
GTCTCGGTCACTTCGACGACCAGATGGCCGAGCGGCACGCGGG
CATGCTCGAACGTGTCGGCCAGCGTGCGTTCGAGCAGGCCGCC
GCCATGGATGTCGGCGGAGCCGACGTTGATCGAGATCTGCGGA
TCGGCGATGCCCAGCCGCATCCAGTGCGCGATGTCGCCGGCGA
CGATCCTCAGCATCCGTCGCGTGAGTTCCGGGGCGATGCGCGG
GTGCGACATCGCTTGGTGGAAGGCGGCGGCCGGCAGCACTTCG
CCCGTGGACGTCGTCAGGCGGCAGAGCGCCTCGAACGACGTTA
CCGCCCATGTCTCCAGTTCGACGACCGGCTGATAATAGGCGTCG
ATACGATCTTCGTGCAGTGCGCGCTCAAGATCGTGCAACGCGTC
GGGATGGCTCGCCACCGCATTGCCCAGGCTCGCGGAATAAGCG
AGGTGGCCGCCGCGGTGCGACTGCTTGGCGTGCTGCAGCGCAT
TGGTGGCATGTTCGAACAGAATGTCGGACGTCTTCGCCGGATC
GCTGATCGCAAAGCCGATG

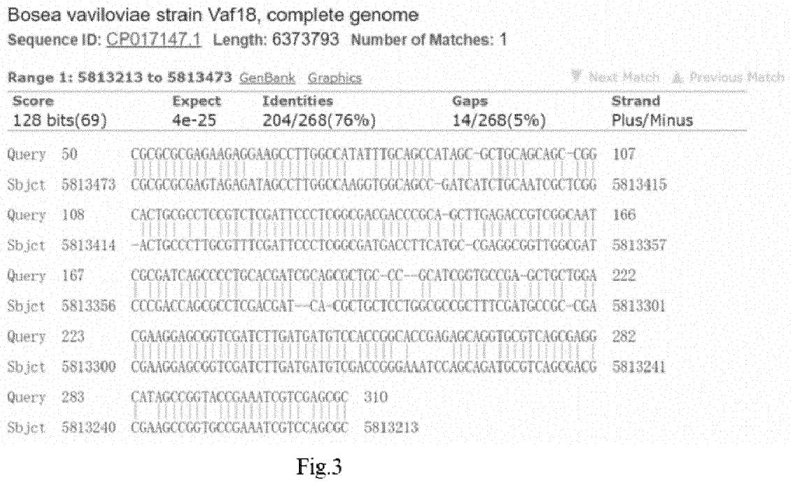

BLASTN alignment is performed between nucleic acid sequences of the specific molecular marker 920 bp of Sanxan gum synthetic strain, and nucleic acid sequences of all species in GenBank database. The result shows that that Bosea vaviloviae strain Vaf18 has the highest homology alignment score, with query cover of 28%. That is, only 28% of the sequence length has comparable homology with that of the molecular marker of Sanxan gum synthetic strain, and the homology is 76%. As shown in FIG. 3, the molecular marker does not have comparable homology sequences with the genomes of gellan gum and welan gum synthetic strains, and the molecular marker of the present invention has high specificity from the perspective of bioinformatics.

### Embodiment 2

Detection of the Molecular Marker of Sanxan Gum Synthetic Strain

The Sanxan gum synthetic strain-Sphingomonas sp. CGMCC No. 1650, the gellan gum synthetic strain, the welan gum synthetic strain, and the xanthan gum synthetic strain as described in Embodiment 1 are respectively inoculated into 250 mL flasks containing 100 mL sterile liquid culture medium; the culture medium comprising (g/L): glucose 12.0; malt extract 4.0; peptone 2.5; yeast powder 1.5; pH=7.0; performing shaking cultivation at 150 rpm, 30° C. in a shaker for 48 hours; centrifuging at 6,000 rpm for 15 minutes; collecting strain cells; and performing genomic DNA extraction by using strain genomic DNA extraction kit from Tiangen Biotech (Beijing) Co., Ltd. PCR amplification is performed by using the primer 1 and primer 2 as primers. The PCR amplification system includes the following components: 1.0 μL of 10-30 ng/μL template DNA, 0.5 μL upstream primer, 0.5 μL downstream primer, 1.0 μL of 10 mM dNTP, 2.5 μL 10× buffer, 0.5 μL of 5 U/μL Platinum Taq DNA polymerase, and water to 25 μL. The PCR reaction conditions includes the following conditions: pre-denaturation at 95° C. for 5 minutes; 94° C. for 45 seconds, 62° C. for 45 seconds, 72° C. for 1 minute, 30 cycles; 72° C. extension for 10 minutes. 5 μL of the PCR amplification product is mixed with 1 μL loading buffer. The mixture is loaded on a 1.5% agarose gel for electrophoresis, stained with GoldView nucleic acid dye for 10 min, and photographed in the gel imager. The results show that the Sanxan gum synthetic strain could be PCR amplified to obtain a clear band around 950 bp. The sequence SEQ13 of the molecular marker is obtained by sequencing. The gellan gum synthetic strain, the welan gum synthetic strain, and the xanthan gum synthetic strain do not appear any amplification band. It indicates that the molecular marker can be used for the identification of Sanxan gum synthetic strain.

### Embodiment 3

The Sanxan gum synthetic strain-Sphingomonas sp. CGMCC No. 19480 is inoculated into a 250 mL flask containing 100 mL sterile liquid culture medium; the culture medium comprising (g/L): glucose 12.0; malt extract 4.0; peptone 2.5; yeast powder 1.5; pH=7.0; performing shaking cultivation at 150 rpm, 30° C. in a shaker for 48 hours; centrifuging at 6,000 rpm for 15 minutes; collecting strain cells; and performing genomic DNA extraction by using strain genomic DNA extraction kit from Tiangen Biotech (Beijing) Co., Ltd. PCR amplification is performed by using the primer 1 and primer 2 as primers. The PCR amplification system includes the following components: 1.0 μL of 10-30 ng/μL template DNA, 0.5 μL upstream primer, 0.5 μL downstream primer, 1.0 μL of 10 mM dNTP, 2.5 μL 10× buffer, 0.5 μL of 5 U/μL Platinum Taq DNA polymerase, and water to 25 μL. The PCR reaction conditions includes the following conditions: pre-denaturation at 95° C. for 5 minutes; 94° C. for 45 seconds, 62° C. for 45 seconds, 72° C. for 1 minute, 30 cycles; 72° C. extension for 10 minutes. 5 μL of the PCR amplification product is mixed with 1 μL loading buffer. The mixture is loaded on a 1.5% agarose gel for electrophoresis, stained with GoldView nucleic acid dye for 10 min, and photographed in the gel imager. The results show that the Sanxan gum synthetic strain-Sphingomonas sp. CGMCC No. 19480 could be PCR amplified to obtain a clear band around 950 bp. The sequence SEQ13 of the molecular marker is obtained by sequencing. It indicates that the molecular marker can be used for the identification of Sanxan gum synthetic strain.

### Embodiment 4

Detection of the Molecular Marker of Sanxan Gum

Sample type: suspension beverage containing Sanxan gum, fruit and vegetable juice (pulp) beverage containing Sanxan gum, and vegetable protein beverage containing Sanxan gum, etc.

The preparation method of citrus suspension beverage containing Sanxan gum includes the following steps: adding the Sanxan gum prepared by the Sanxan gum synthetic strain in Embodiment 1 into water at a mass volume percentage of 0.05%, stirring for 12 h, fully dissolving, heating to 90° C. and maintaining for 15 min, cooling to 50° C. and adding citrus vesicle at a mass volume percentage of 6%, adding white granulated sugar at a mass volume percentage of 5%, adjusting the pH of the solution to 4.0 with 1 mol/mL citric acid, bottling, sterilizing at 121° C. for 15min, cooling, shaking, standing for 48 h to obtain citrus suspension beverage, filtering the above citrus suspension beverage containing Sanxan gum with a 200-mesh filter screen, centrifuging the clear liquid at 43000× g for 30 min, repeating the above operation to enrich the precipitate to 10 g, performing genomic DNA extraction according to the instructions of genome large-scale extraction kit of Beijing Solarbio Science & Technology Co., Ltd., and performing electrophoresis detection. The PCR amplification is performed by using the extracted genomic DNA as a template, where the PCR amplification system and reaction conditions are the same as in Embodiment 1. The PCR amplification band around 950 bp detected by electrophoresis is subjected to DNA sequencing in Beijing Aoke Dingsheng Biotechnology Co., Ltd., and the sequences near primers that affect the accuracy of sequencing are removed to obtain specific molecular marker SEQ13 representing Sanxan gum.

Therefore, the specific molecular marker of the present invention can be used for identifying Sanxan gum in mediumly and lightly processed products containing the Sanxan gum.

### Embodiment 5

Detection of the Molecular Marker

The preparation method of citrus suspension beverage containing gellan gum includes the following steps: adding the gellan gum prepared by the gellan gum synthetic strain in Embodiment 1 into water at a mass volume percentage of 0.05%, stirring for 12 h, fully dissolving, heating to 90° C. and maintaining for 15 min, cooling to 50° C. and adding citrus vesicle at a mass volume percentage of 6%, adding white granulated sugar at a mass volume percentage of 5%, bottling, sterilizing at 121° C. for 15min, cooling, shaking, and standing for 48 h to obtain citrus suspension beverage containing gellan gum.

The preparation method of citrus suspension beverage containing welan gum includes the following steps: adding the welan gum prepared by the welan gum synthetic strain in Embodiment 1 into water at a mass volume percentage of 0.05%, stirring for 12 h, fully dissolving, heating to 90° C. and maintaining for 15 min, cooling to 50° C. and adding citrus vesicle at a mass volume percentage of 6%, adding white granulated sugar at a mass volume percentage of 5%, bottling, sterilizing at 121° C. for 15min, cooling, shaking, and standing for 48 h to obtain citrus suspension beverage containing welan gum.

The genomic DNA extraction method is the same as in Embodiment 3, and the PCR amplification system and reaction conditions are the same as in Embodiment 2. The results of electrophoresis detection show that no band is amplified from the citrus suspension beverage containing gellan gum and welan gum, and it indicates that the molecular marker of the present invention is specific.

### Embodiment 6

Detection of the Molecular Marker

The preparation method of citrus suspension beverage containing compound gum includes the following steps: adding the Sanxan gum prepared by the Sanxan gum synthetic strain in Embodiment 1 at a mass volume percentage of 0.03%, and the gellan gum prepared by the gellan gum synthetic strain in Embodiment 1 at a mass volume percentage of 0.02% into water, stifling for 12 h, fully dissolving, heating to 90° C. and maintaining for 15 min, cooling to 50° C. and adding citrus vesicle at a mass volume percentage of 6%, adding white granulated sugar at a mass volume percentage of 5%, adjusting the pH of the solution to 4.0 with 1 mol/mL citric acid, bottling, sterilizing at 121° C. for 15min, cooling, shaking, and standing for 48 h to obtain citrus suspension beverage.

The genomic DNA extraction method is the same as in Embodiment 3, and the PCR amplification system and reaction conditions are the same as in Embodiment 2. The PCR amplification band around 950 bp detected by electrophoresis is subjected to DNA sequencing in Beijing Aoke Dingsheng Biotechnology Co., Ltd., and the sequences near primers that affect the accuracy of sequencing are removed to obtain specific molecular marker SEQ13 representing Sanxan gum.

It shows that the molecular marker of the present invention can be used for identifying Sanxan gum in compound gum containing Sanxan gum.

### Embodiment 7

Detection of the Molecular Marker: Edible Film Containing Sanxan Gum

The preparation method of edible film containing Sanxan gum includes the following steps: adding the Sanxan gum prepared by the Sanxan gum synthetic strain in Embodiment 1 into water at a mass volume percentage of 1%, magnetically stirring at 50° C. for 12 h, adding sodium trimetaphosphate at a mass percentage of 0.1%, performing cross-linking reaction at 25° C. and pH of 7.5 for 1 h, adding the same volumes of 1% sodium alginate solution and 0.7% glycerol to the cross-linked modified Sanxan gum solution to obtain a film solution, and applying it to the surface of strawberries and other fruits for freshness. Rinse the above strawberries containing edible films of Sanxan gum repeatedly with tap water of 0.5 times of weight for 30min, stir the rinsed film liquid at room temperature on a magnetic stirrer for 12 h, filter with a 200-mesh filter screen, centrifuge the clear liquid at 43000× g for 30 min, repeat the above operation to enrich the precipitate to 10 g, perform genomic DNA extraction according to the instructions of genome large-scale extraction kit of Beijing Solarbio Science & Technology Co., Ltd., and perform electrophoresis detection. The PCR amplification system and reaction conditions are the same as in Embodiment 1. The PCR amplification band around 950 bp detected by electrophoresis is subjected to DNA sequencing in Beijing Aoke Dingsheng Biotechnology Co., Ltd., and the sequences near primers that affect the accuracy of sequencing are removed to obtain specific molecular marker SEQ13 representing Sanxan gum. It shows that the molecular marker of the present invention can be used for identifying Sanxan gum in edible film containing Sanxan gum.

What is claimed is:

1. A molecular marker for identifying Sanxan gum and its synthetic strain, being characterized in that, the molecular marker is a nucleotide sequence shown in SEQ No.13.

2. The molecular marker according to claim 1, being characterized in that, a pair of primers is used to obtain the nucleotide sequence shown in SEQ No.13.

3. The molecular marker according to claim 2, being characterized in that, the pair of primers is composed of a primer 1 and a primer 2, wherein the primer 1 is a nucleotide sequence shown in SEQ No. 5, or a nucleotide sequence obtained by deleting, and/or adding, and/or changing at least one nucleotide of the nucleotide sequence shown in SEQ No. 5;
and the primer 2 is the nucleotide sequence shown in SEQ No. 6, or a nucleotide sequence obtained by deleting, and/or adding, and/or changing at least one nucleotide of the nucleotide sequence shown in SEQ No. 6.

4. The molecular marker according to claim 3, being characterized in that, the molecular marker is prepared by the following specific steps:
i), performing PCR reaction using genomic DNA of Sphingomonas sp. with deposit number CGMCC No. 1650 or CGMCC No. 19480 as a template for PCR amplification, and the primer 1 and the primer 2 as primers;
ii), PCR amplification system containing 1.0 μL of 10-30 ng/μL template DNA, 0.5 μL of 10 μM primer 1, 0.5 μL of 10 μM primer 2, 1.0 μL of 10 mM dNTP, 2.5 μL 10× buffer, 0.5 μL of 5 U/μL Platinum Taq DNA polymerase, and ultrapure water to 25μL;
PCR reaction conditions containing pre-denaturation at 95° C. for 5 minutes; 94° C. for 45 seconds, 55-63° C. for 45 seconds, 72° C. for 1 minute, 30 cycles; 72° C. extension for 10 minutes;
a PCR amplification product being about 950 bp by electrophoresis detection;
iii), performing DNA sequencing of the PCR amplification product to obtain a core conserved sequence 920 bp representing Sanxan gum and its synthetic strain by removing sequences of primers at two ends that affect the accuracy of sequencing, which is a specific molecular marker for identifying Sanxan gum and its synthetic strain.

5. A kit for identifying Sanxan gum and its synthetic strain comprising the pair of primers according to claim 3, control DNA templates and PCR amplification system, wherein the PCR amplification system comprises dNTP, Taq DNA polymerase and buffers.

6. The kit according to claim 5, being characterized in that, the synthetic strain of Sanxan gum is Sphingomonas sp. having a deposit number of CGMCC No. 1650 or CGMCC No. 19480.

7. A method for identifying Sanxan gum and its synthetic strain by the molecular marker according to claim 1, wherein mediumly or lightly processed products for identifying Sanxan gum containing a synthetic strain of Sanxan gum.

8. The method according to claim 7, being characterized in that, the synthetic strain of Sanxan gum is Sphingomonas sp. having a deposit number of CGMCC No. 1650 or CGMCC No. 19480.


SEQUENCE LISTING (13 sequences)

SEQ ID NO: 1 - DNA, 20 bp, Artificial Sequence
  It is synthesized
tgaggtcgaggaacttgtgc

SEQ ID NO: 2 - DNA, 20 bp, Artificial Sequence
  It is synthesized
gtcgtgaccgactatcgcat

SEQ ID NO: 3 - DNA, 20 bp, Artificial Sequence
  It is synthesized
aggtcgaggaacttgtgcag

SEQ ID NO: 4 - DNA, 20 bp, Artificial Sequence
  It is synthesized
tcgccaccaagatctatcgc

SEQ ID NO: 5 - DNA, 17 bp, Artificial Sequence
  It is synthesized
tcaggccgtgtggggaa

SEQ ID NO: 6 - DNA, 20 bp, Artificial Sequence
  It is synthesized
gatccgatccagctttcggg

SEQ ID NO: 7 - DNA, 20 bp, Artificial Sequence
  It is synthesized
cagaatgctcgtattgccgc

SEQ ID NO: 8 - DNA, 20 bp, Artificial Sequence
  It is synthesized
tctatctcgacgagcgcaac

SEQ ID NO: 9 - DNA, 20 bp, Artificial Sequence
  It is synthesized
ggcttcggtgaagaacagga

SEQ ID NO: 10 - DNA, 20 bp, Artificial Sequence
  It is synthesized
tcgatccaccatatgcagcc

SEQ ID NO: 11 - DNA, 22 bp, Artificial Sequence
  It is synthesized
cttggccatatttgcagccata

SEQ ID NO: 12 - DNA, 20 bp, Artificial Sequence
  It is synthesized
gaccggatttgctcagggtt

SEQ ID NO: 13 - DNA, 920 bp, Artificial Sequence
  It is synthesized
acggcaggacctcgccttgcagcagccgcgtcgcctggcgacggtcgagcgcgcgcgagaagaggaagccttggccatatttgcagccatagcgctgcagcagccggcactgcgcctccgtctcgattccctcggcgacgacccgcagcttgagaccgtcggcaatcgcgatcagcccctgcacgatcgcagcgctgcccgcatcggtgccgagctgctggacgaaggagcggtcgatcttgatgatgtccaccggcaccgagagcaggtgcgtcagcgaggcatagccggtaccgaaatcgtcgagcgcgatgcggagcccgcgcgcctgcaatccttcgagaacgcggcgcacggtatcggcgcgccggtccatatggaccgtctcggtcacttcgacgaccagatggccgagcggcacgcgggcatgctcgaacgtgtcggccagcgtgcgttcgagcaggccgccgccatggatgtcggcggagccgacgttgatcgagatctgcggatcggcgatgcccagccgcatccagtgcgcgatgtcgccggcgacgatcctcagcatccgtcgcgtgagttccggggcgatgcgcgggtgcgacatcgcttggtggaaggcggcggccggcagcacttcgcccgtggacgtcgtcaggcggcagagcgcctcgaacgacgttaccgcccatgtctccagttcgacgaccggctgataataggcgtcgatacgatcttcgtgcagtgcgcgctcaagatcgtgcaacgcgtcgggatggctcgccaccgcattgcccaggctcgcggaataagcgaggtggccgccgcggtgcgactgcttggcgtgctgcagcgcattggtggcatgttcgaacagaatgtcggacgtcttcgccggatcgctgatcgcaaagccgatg